(19) United States
(12) Patent Application Publication
(10) Pub. No.: US 2012/0165886 A1
(43) Pub. Date: Jun. 28, 2012

(54) ELECTRICAL STIMULATION DEVICE FOR TREATING CARDIOVASCULAR DISEASE AND METHOD FOR TREATING CARDIOVASCULAR DISEASE

(75) Inventors: Kenji Sunagawa, Fukuoka (JP); Tomomi Ide, Fukuoka (JP); Makoto Ando, Fukuoka (JP)

(73) Assignee: Kyushu University, National University Corporation, Fukuoka (JP)

(21) Appl. No.: 13/257,583

(22) PCT Filed: Mar. 19, 2010
(86) PCT No.: PCT/JP2010/002016

§ 371 Date: Feb. 3, 2012

Related U.S. Application Data
(60) Provisional application No. 61/161,456, filed on Mar. 19, 2009.

(51) Int. Cl. A61N 1/362

(52) U.S. Cl. 607/9

(57) ABSTRACT

The object of the invention is to provide a new treatment method and treatment device that together with being able to improve a drop in cardiac contractility when treating cardiovascular disease such as acute myocardial infarction and suppress the occurrence of arrhythmia, is able to reduce the infarct size. Provided is an electrical stimulation device for treating cardiovascular disease having at least one electrode that is placed on a nerve site in the body of an animal, and an electrical stimulation application unit that applies electrical stimulation by the electrode to the vagus nerve in the neck region of the animal; and a method for treating cardiovascular disease in an animal having a step of placing an electrode in contact with the vagus nerve in the neck region of an animal, and a step of applying electrical stimulation to the animal.

### CROSS REFERENCE TO RELATED APPLICATIONS

This application claims priority under 35 U.S.C. 119 based upon U.S. provisional application No. 61/161,456, filed on Mar. 19, 2009. The entire disclosures of the aforesaid applications are incorporated herein by reference.

### FIELD OF THE INVENTION

The present invention relates to an electrical stimulation device and treatment method for treating animals with cardiovascular disease.

More specifically, the electrical stimulation device for treating cardiovascular disease of the present invention has an electrode and an electrical stimulation application unit that applies electrical stimulation to the vagus nerve in the neck region of an animal, and is able to treat cardiovascular disease by applying electrical stimulation to the vagus nerve. Moreover, in the treatment method for cardiovascular disease of the present invention, an electrode is placed such as to come in direct contact with the vagus nerve in the neck region of an animal, and cardiovascular disease is treated by applying electrical stimulation to the vagus nerve by way of this electrode.

### BACKGROUND OF THE INVENTION

Myocardial infarction is a kind of ischemic heart disease, and is a state in which the amount of blood flow in coronary artery as nutrition to the heart drops, the heart muscle becomes ischemic and the heart dies. Normally, this refers to an acutely occurring “acute myocardial infarction (AMI)”. The method of treatment during the acute phase is as a rule complete rest. During the acute phase after the onset of disease, it is easy for lethal arrhythmia to occur, and the danger of dying is extremely high. The more the ischemic period is prolonged, the more death of the heart muscle advances, and an irreversible decrease of cardiac performance occurs. When disease is first suspected, it is necessary to immediately call an ambulance while keeping an eye on the patient, and in the case that the patient become unconscious and there is no pulse, it becomes necessary to perform heart massage without hesitation. When functional cardiac arrest occurs, going three to five minutes or more without performing treatment results in a rehabilitation rate of nearly zero. It is necessary to start emergency treatment (heart massage or the like) without waiting for the ambulance to arrive.

Myocardial infarction is caused by insufficient relative and absolute oxygen supply to the heart muscle; and as a method of treatment, the patient is kept quiet in bed and oxygen inhalation is performed. In some cases morphine may also be administered in order to relieve pain and reduce oxygen consumption. The main objective during the acute phase is to prevent lesion expansion of myocardial infarction. Generally, the treatment performed as first aid for myocardial infarction is centered on “oral administration of aspirin”, “oxygen inhalation”, “administration of morphine” and “administration of nitrate”, and is known by the name “MONA”, taking the first letter from Morphine, Oxygen, Nitrate and Aspirin.

By actively performing reperfusion therapy of the obstructed coronary artery within six hours or less from the onset of the myocardial infarction, it is possible to reduce the range of necrosis of the heart muscle. Not being limited to this, in an example of a case within 24 hours from the onset of the illness, performing reperfusion therapy is highly meaningful. Generally, treatment may be divided into the case of performing catheter therapy (PTCA, PCI), or thrombolytic therapy (PTCR), and different treatment policies are adapted depending on the country, insurance or doctor's judgment. In Japan, many facilities are capable of performing PCI, and in many cases, PCI is performed during the acute phase. However, because examination and treatment are performed via an artery, complications often occur. Particularly, when a rise in ST is seen on the electrocardiogram, it is essential that PCI be performed as soon as possible, however, there are a few hospitals, even in the USA, which is a leading nation in the treatment of heart disease, that take the position of performing the same treatment immediately after the patient has been admitted to emergency. In the case of there being three or more sites of stenosis, there are some facilities that will perform emergency coronary artery bypass graft surgery (CABG). In comparing PCI and CABG, for PCI restenosis occurs in 25 to 30% of cases, so even in the case of single-vessel disease, there are cases where CABG has advantages. However, since 2004, drug-eluting stents (DES) are covered by insurance, so an improvement in the result of PCI treatment is expected. When intervention is successful during the acute phase, relative prognosis is often maintained. Intervention is one method for treating illness of the heart, blood vessels, the liver, the brain, digestive organs, urinary organs and the like, and is mainly a treatment method for performing treatment by inserting a small tube called a catheter into a blood vessel from a small hole having a diameter of several mm that is made in the skin. Intervention is a method of treatment that has very little burden on the patient, and recently has attracted much attention. The cut is small, so recovery after surgery is fast, and after a very short hospital stay of three to five days, together with greatly improving the QOL (Quality of Life) of the patient, this treatment reduces the financial burden on the patient, and is said to even contribute to health care cost-containment measures by the government. However, in reperfusion therapy such as intervention, complications such as arrhythmia, extrasystole, ventricular fibrillation, atrioventricular block or heart failure often occur.

Quick cardiovascular recovery is essential for maintaining life, however, exposes oneself to danger. Reperfusion increases localized damage, and produces an inflammatory reaction that also leads to systemic insult. Acute onset of myocardial infarction, stroke, cardiac arrest and the like can produce ischemia-reperfusion injury (IRI). However, many scheduled surgical treatments such as organ transplants and aneurysm treatment require a period of ischemia between treatments, and therefore may produce the onset of IRI. Conventionally it was thought that the existence of inflammatory cells in the ischemic tissue indicated a pathophysiological response to injury. However, according to laboratory tests, it has been shown that inflow to inflammatory cells, and particularly to macrophage tissue, which is a phagocyte, even though important for recovery, also brings about tissue damage that exceeds the tissue damage caused by ischemia alone. This damage can have an effect on various kinds of tissue such as the heart, brain, liver, spleen, intestines, lungs and pancreas.

Various methods for putting an end to reperfusion injury such as induced hypothermia, controlled reperfusion, ischemic preconditioning and the like have been reported. Induced hypothermia is the introduction of moderately low temperature (28° C. to 32° C.) to a patient. Mild induced hypothermia is thought to suppress many chemical reactions related to reperfusion injury. Regardless of these potential benefits, induced hypothermia also brings about side effects such as arrhythmia, infection, blood clotting and the like. Controlled reperfusion means to control the initial stage of reperfusion by performing reperfusion of tissue at low pressure using blood that has been altered so that there is hyperosmosis, alkalosis, and substrate concentration. Ischemic preconditioning is intentionally causing short-term ischemia, which has a protective effect, to occur by slowing down cellular metabolism between the onset of more prolonged ischemia. These treatments are useful in a surgical setting (for example, before or after scheduled heart surgery), however, normally, these treatments are not suitable in a preset condition that is controlled as required.

In recent years, applying electrical stimulation to the vagus nerve has been reported as an effective treatment method for chronic heart failure. In other words, when electrical stimulation of the vagus nerve is performed, the heart rate drops, and as the heart rate drops, the myocardial oxygen consumption is reduced, and a state of oxygen deprivation in the heart muscle is prevented or improved. As a result, the occurrence of myocardial ischemia and the accompanying lethal arrhythmia are prevented, so this method is considered to be effective as treatment for or prevention of heart failure. Technology has been disclosed related to a vagus nerve stimulation system for performing electrical stimulation of the vagus nerve, and particularly, technology has been disclosed related to a vagus nerve stimulation system that is capable of indirectly stimulating the vagus nerve from under the skin or from the surface of the skin (Japanese Patent Application laid-open publication No. 2005-500863, Japanese Patent Application laid-open publication No. 2009-233024).

### SUMMARY OF THE INVENTION

Considering the above situation, the purpose of the present invention is to provide a new treatment method and treatment device that are capable of improving a drop in myocardial contractility, suppressing the occurrence of arrhythmia, and reducing the size of infarction when treating cardiovascular disease such as acute myocardial infarction.

The inventors took notice of the effect of treating cardiovascular disease by applying electrical signals to nerves, and as a result of dedicated investigation, completed the present invention.

More specifically, the present invention provides a treatment method and treatment device for treating cardiovascular disease by applying electrical stimulation to the vagus nerve in the neck region of an animal under specified conditions. By applying electrical stimulation to the neck region under certain conditions in this way, a superior treatment effect that prevents the occurrence of the above mentioned complications during treatment of cardiovascular disease becomes possible. By using the present invention to perform treatment by vagus nerve stimulation during emergency transport, it is possible to significantly lower the occurrence of complications after ischaemia reperfusion.

According to a first major aspect of the present invention, an electrical stimulation device for treating cardiovascular disease is provided that comprises at least one electrode that is placed on a nerve site inside an animal, and an electrical stimulation application unit that applies electrical stimulation to the vagus nerve in the neck region of the animal by the electrode.

According to an embodiment of the present invention the electrical stimulation device for treating cardiovascular disease can be applied to treatment of acute myocardial infarction. The electrical stimulation device for treating cardiovascular disease of the present invention displays an excellent effect in the treatment of cardiovascular disease, particularly in the treatment of acute myocardial infarction.

Moreover, according to another embodiment of the present invention, the conditions for applying electrical stimulation when treating cardiovascular disease with the electrical stimulation can be a voltage of 0.1 to 5 V, a frequency of 1 to 30 Hz and a pulse width of 500 μsec. By applying electrical stimulation to the vagus nerve in the neck region under these conditions, it is possible to prevent complications that accompany treatment, and to obtain excellent therapeutic effect.

Furthermore, with another embodiment of the invention, the amount of time that electrical stimulation is applied using the electrical stimulation application unit can be at least 0.5 hour and no greater than 10 hours. By applying electrical stimulation for an amount of time within this range, effective treatment of cardiovascular disease is possible.

According to a second major aspect of the present invention, a method for treating cardiovascular disease in an animal is provided that comprises steps of: placing an electrode in contact with the vagus nerve in the neck region of an animal; and applying an electrical stimulation by the electrode.

According to an embodiment of the present invention, the treatment method of the invention can be applied to treating cardiovascular disease of humans. Moreover, according to another embodiment of the present invention, the treatment method of the invention can be applied to treating cardiovascular disease of animals other than humans.

According to another embodiment of the present invention, the method for treating cardiovascular disease of this invention can be applied when the cardiovascular disease is acute myocardial infarction. The method for treating cardiovascular disease of the present invention particularly displays an effect when the cardiovascular disease is acute myocardial infarction.

Moreover, according to another embodiment of the present invention, it is possible for the step of applying an electrical stimulation of this method for treating cardiovascular disease to be used in combination with reperfusion therapy. By using the treatment method of this invention in combination with reperfusion therapy, even more effective treatment of cardiovascular disease can be expected. The step of applying an electrical stimulation of the present invention can be performed after the occurrence of acute myocardial infarction and before performing reperfusion therapy.

Furthermore, according to another embodiment of the present invention the conditions of a voltage of 0.1 to 5 V, a frequency of 1 to 30 Hz, and a pulse width of 500 μsec or greater can be applied as the conditions for applying electrical stimulation in the method for treating cardiovascular disease of this invention. By applying electrical stimulation under these conditions, it is particularly possible to perform effective treatment with no adverse effects such as complications.

### BRIEF DESCRIPTION OF THE DRAWINGS

FIG. 1 illustrates the test protocol of an embodiment of the present invention.

FIG. 2 is a graph illustrating the change in heart rate during stimulation of the vagus nerve in an embodiment of the present invention.

FIG. 3 is a drawing illustrating the infarction area that is suppressed by stimulation of the vagus nerve in an embodiment of the present invention.

FIG. 4 is a table illustrating the hemodynamic change four days after ischemia reperfusion.

FIG. 5 is a drawing illustrating the ratio of TUNEL staining in the infarction area 24 hours after ischemia.

FIG. 6 is a drawing illustrating the expression of mRNA related to the production of collagen in the infarction site (measured using the real time PCR method).

FIG. 7 is a drawing illustrating the site of electrical stimulation in an embodiment of the present invention.

FIG. 8 is a drawing suggesting that, because the heart rate dropped when an existing magnetic stimulation device was applied to the neck region of a dog, the vagus nerve was stimulated by an electric field that was generated by generating a magnetic field.

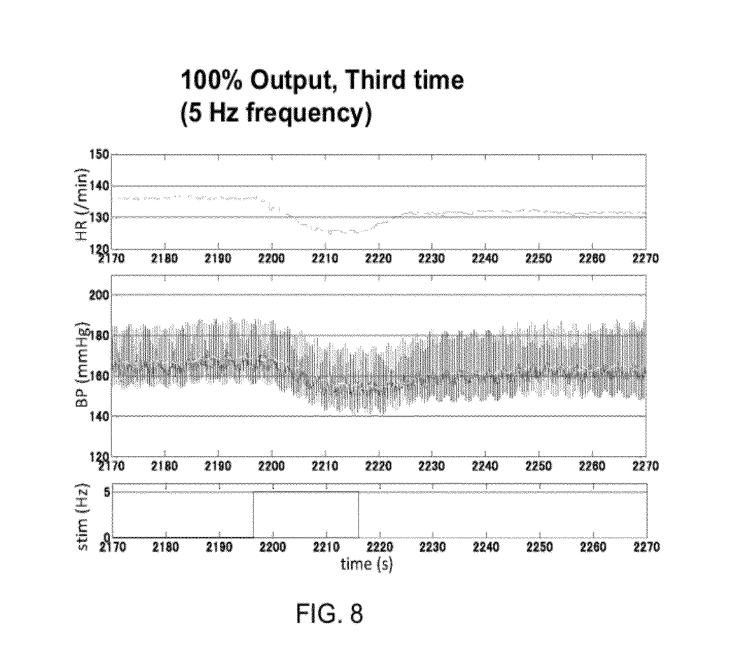

FIG. 9 is a drawing illustrating an example of a magnetic stimulation device that can be applied to the present invention.

### DETAILED DESCRIPTION OF THE INVENTION

In the following, the present invention will be described in detail. Any kind of device can be used as the electrical stimulation device for treating cardiovascular disease of the present invention as long as it is a device having at least one electrode that can be placed at the site of a nerve, and has an electrical stimulation unit for applying electrical stimulation to the vagus nerve of an animal by way of the electrode. The electrical stimulation unit can comprise, for example, a DC voltage generating circuit that generates a specified voltage, a capacitor that is charged by voltage that is generated by the DC voltage generating circuit, and a switch that is located between the capacitor and the electrode and that is for switching ON/OFF a connection between the capacitor and electrode.

In the electrical stimulation device for treating cardiovascular disease of the present invention, the electrode applies electrical stimulation by coming in direct contact with the vagus nerve of an animal. Here, the vagus nerve refers mainly to a parasympathetic nerve that controls the internal organs of the thoracicoabdominal region, and also involved in adjustment of the heart rate, peristaltic movement of the stomach and intestines, perspiration, and speech. This vagus nerve runs from the brain stem to the abdominal region.

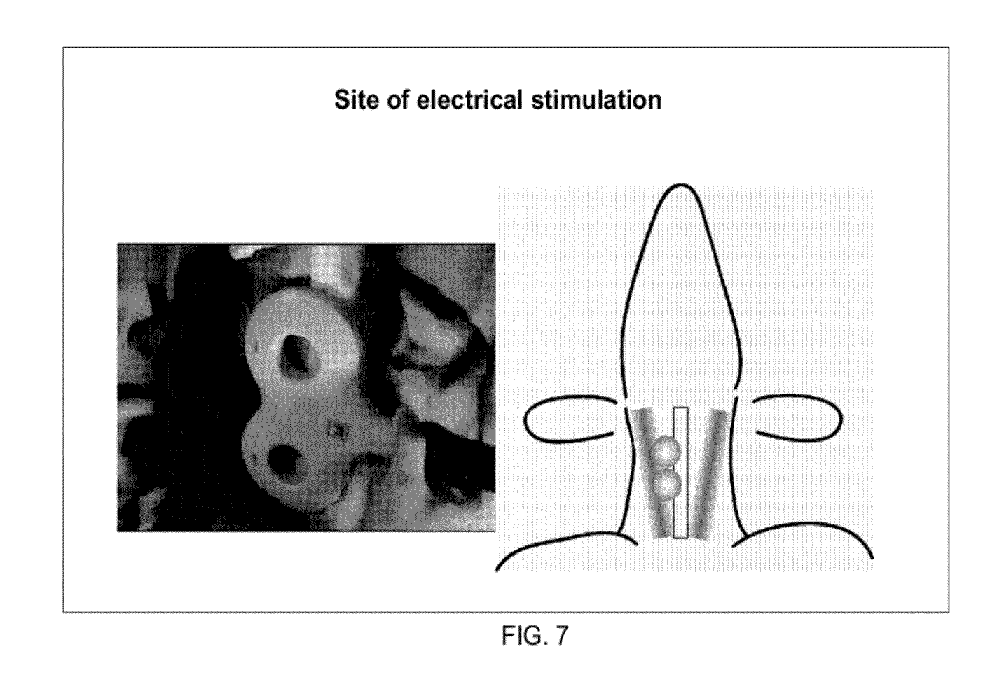

In the present invention, the site of stimulation by the electrode is not particularly limited as long as it on a site of the vagus nerve, however, preferably the electrical stimulation is applied to the vagus nerve in the neck region. FIG. 7 illustrates the electrical stimulation site in the neck region in an embodiment of the present invention. When electrical stimulation is applied to a site of the vagus nerve by the electrode, the site of the vagus nerve to which the electrical stimulation is applied can be peeled back and exposed, for example, and the electrical stimulation can be applied by bringing the electrode in direct contact with the site. It is also possible to apply acupuncture stimulation to an acupuncture point that stimulates the vagus nerve, or apply electrical stimulation from inside a blood vessel.

The electrical stimulation device for treating cardiovascular disease of the present invention can be constructed with only an electrode and electrical stimulation application unit as described above, so from the aspect of compactness and convenience of operation, treatment by stimulation of the vagus nerve can even be performed during emergency transport. By being able to perform treatment by stimulation of the vagus nerve during emergency transport, it becomes possible to effectively lower the occurrence of complications after ischemia reperfusion.

The electrical stimulation device for treating cardiovascular disease of the present invention can be applied to various kinds of cardiovascular disease that are particularly treatable by stimulating the vagus nerve. For example, the device can be applied to treatment such as acute myocardial infarction, angina including unstable angina, heart failure, arrhythmia, hypertension, arteriosclerosis, and the like, and is particularly effective in the treatment of acute myocardial infarction and heart failure.

Conditions for applying electrical stimulation using the electrical stimulation device for treating cardiovascular disease of the present invention can be suitably set according to conditions that meet the severity of the patient's condition, and can be set within the range: a voltage of 0.01 to 20 V, frequency of 0.1 to 40 Hz, and pulse width of 500 μsec or greater. For example, in an embodiment of the present invention, conditions of a voltage of 0.1 to 5 V, frequency of 1 to 30 Hz, and pulse width of 500 μsec or greater are applied.

The time that electrical stimulus is applied in the present invention can be appropriately selected to correspond to the severity of the patient, however should be selected between 0.1 to 10 hours, or more preferably between 0.5 to 10 hours. The electrical stimulus can be applied intermittently at fixed time intervals, or can be applied continuously for a fixed time. After electrical stimulation has been applied for a fixed time, electrical stimulation can be applied again in the case that disease such as arrhythmia occurs.

Moreover, the method for treating cardiovascular disease of the present invention can be performed in combination with reperfusion therapy. When performed in combination with reperfusion therapy, the application of electrical stimulation can be performed before reperfusion therapy, can be performed at the same time as reperfusion therapy or can be performed after reperfusion therapy, however, preferably the electrical stimulation of the present invention is performed before performing the reperfusion therapy.

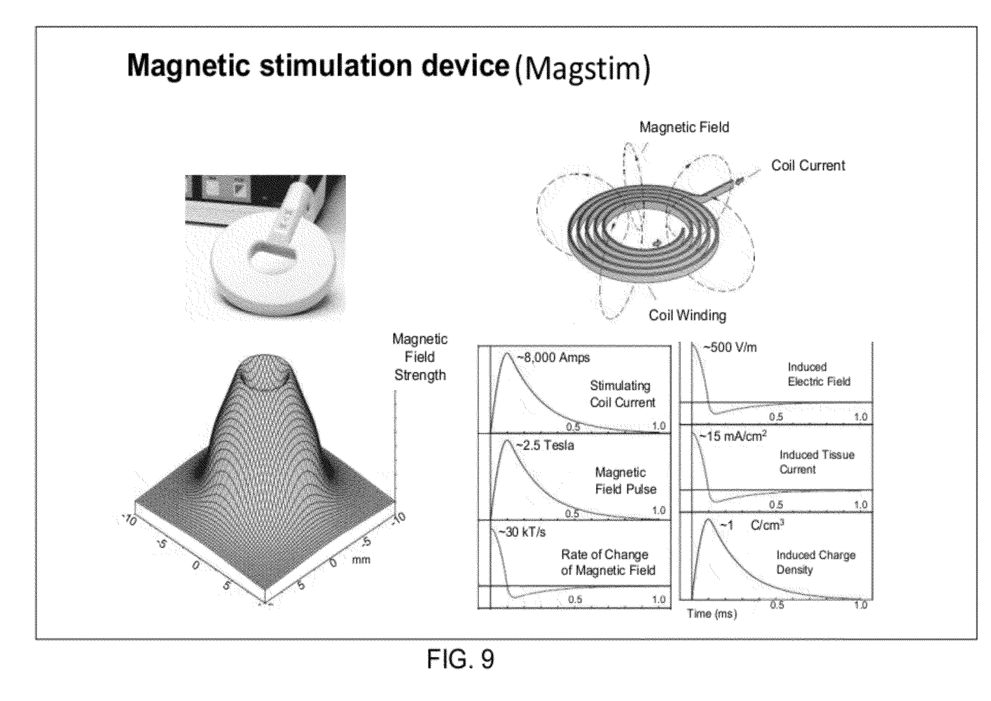

Application of electrical stimulation is performed as the method for treating cardiovascular disease in the present invention, however, in addition to this, magnetic stimulation can also be used. For example, transcranial magnetic stimulation is performed using a magnetic stimulator that uses a coil for treating depression, however, by using a magnetic stimulation device to apply magnetic stimulation to the neck area of an animal, the same effects of lowering the heart rate and stimulating the vagus nerve can be obtained (FIG. 9).

Next, the effect of the present invention will be explained by illustrating an embodiment. However, the present invention is not limited to the embodiment described below, and it is understood that various changes and modification can be easily performed by one skilled in the art.

### Embodiment 1

Thoracotomy was performed under anesthesia for a male SD rat, the left coronary artery was ligated, and after 30 minutes of ischemia, reperfusion was performed by loosening the ligature, which created myocardial infarction (MI). The right neck region was peeled to expose the vagus nerve, and stimulation of the vagus nerve was performed under the conditions of a 0 to 3V voltage, 1 msec pulse width and 5 Hz frequency so that a heart rate that was lowered by about 10% was obtained. Vagus never stimulation (VS), including reperfusion, was performed for 30 minutes from the time of ischemia, and after 24 hours, evaluations of the infarct area and apoptosis were performed, and after 4 days, evaluation of the hemodynamics was performed.

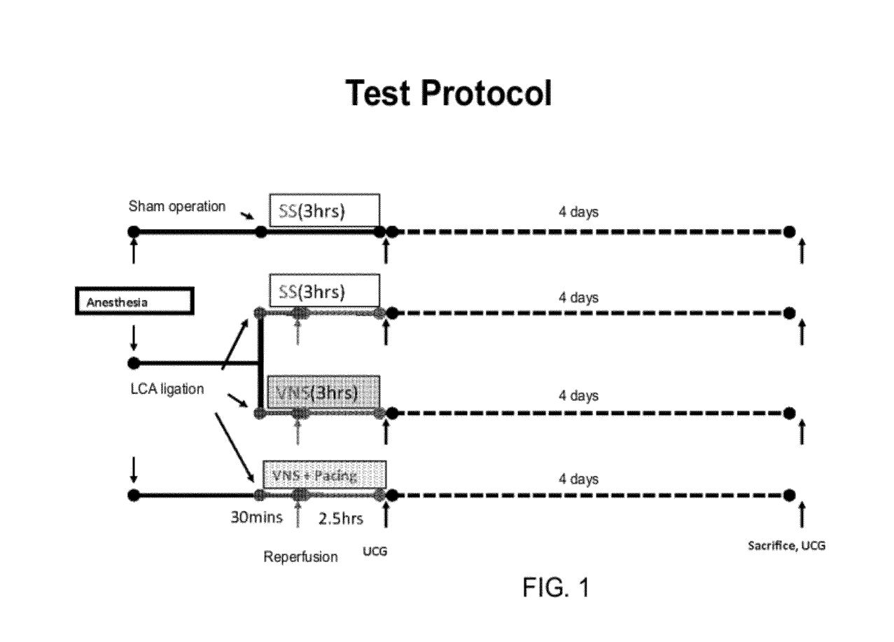

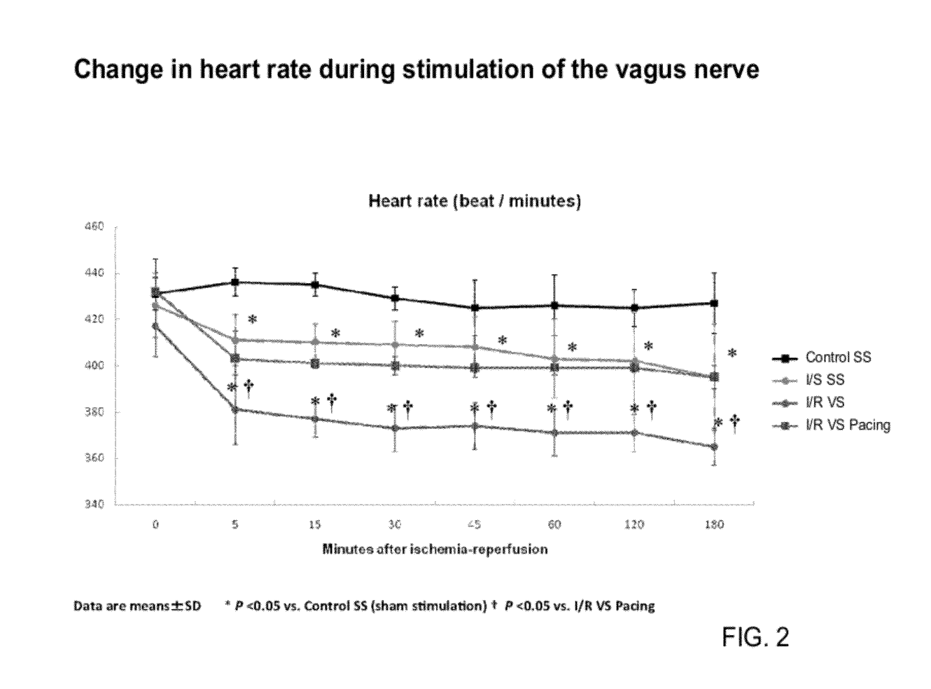

As a control, a group was made in which only a thoracotomy was performed, and as sham stimulation (SS), only an electrode was mounted without applying current. Moreover, in order to study the effect of bradycardia that occurs as the heart rate drops due to stimulation of the vagus nerve, pacing (VNS+pacing) was performed electrically in the right atrium during stimulation of the vagus nerve so that the heart rate could be maintained the same as in the SS group. The experimental protocol described above is illustrated in FIG. 1, and the change in the heart rate during stimulation of the vagus nerve is illustrated in FIG. 2.

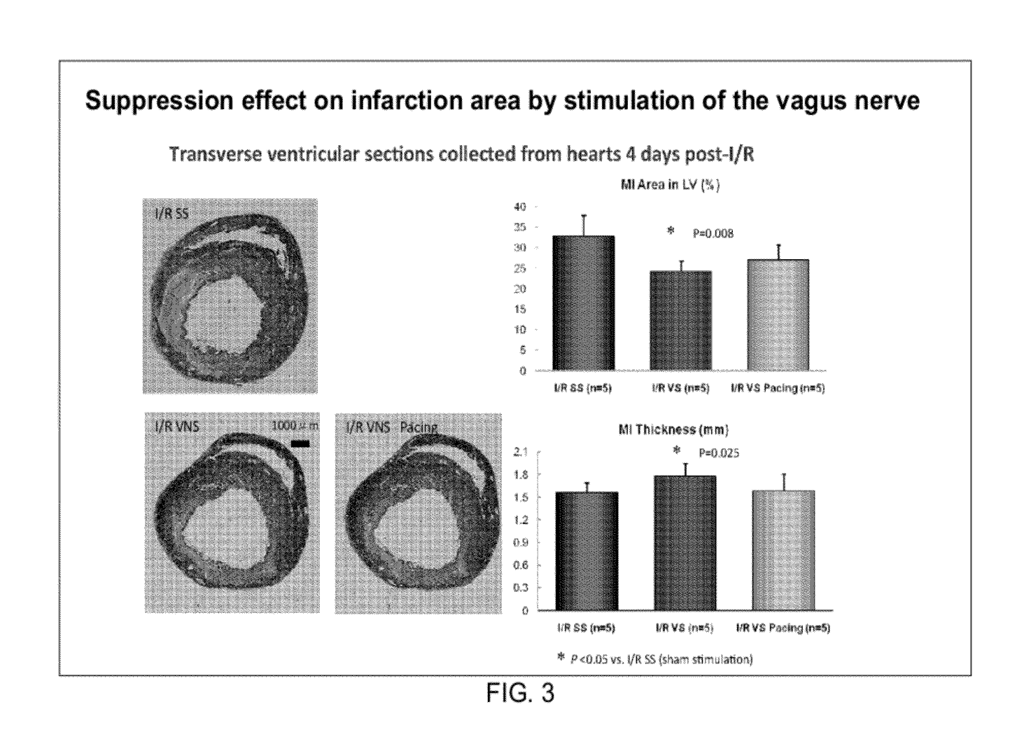

It became clear from analysis after four days that during stimulation of the vagus nerve, the infarct area was significantly reduced. That result was partially reversed by pacing (FIG. 3). The results of measuring the hemodynamics four days later are illustrated in Table 1.

TABLE 1
Change in Hemodynamics 4 Days After Ischaemia Reperfusion
Electrocardiogram Data (Under Anesthesia)

| | | | | |
|---|---|---|---|---|
| Heart rate (bbp) | 417 ± 17 | 415 ± 7 | 417 ± 12 | 424 ± 11 |
| Left ventricular end-diastolic | 5.4 ± 0.3 | 7.5 ± 0.5 * | 6.6 ± 0.5 * † | 6.6 ± 0.7 * |
| dimension (mm) | | | | |
| Left ventricular end-systolic | 2.6 ± 0.2 | 5.9 ± 0.6 * | 4.4 ± 0.6 * † | 5.1 ± 0.6 * |
| dimension (mm) | | | | |
| Ejection fraction (%) | 51.5 ± 3.2 | 21.6 ± 4.2 * | 31.7 ± 6.2 * † | 23.9 ± 2.6 * |
| Infarct wall thickness (mm) | 1.9 ± 0.2 | 1.3 ± 0.2 * | 1.4 ± 0.2 * | 1.3 ± 0.2 * |
| Non-infarct wall thickness (mm) | 2.3 ± 0.3 | 1.9 ± 0.2 * | 2.0 ± 0.2 | 1.9 ± 0.1 * |

Catheter Measurement Value (Under Anesthesia)

| | | | | |
|---|---|---|---|---|
| Heart rate (bpm) | 419 ± 8 | 409 ± 7 | 410 ± 14 | 424 ± 11 |
| Blood pressure (mmHg) | 119 ± 4 | 100 ± 5 * | 102 ± 8 * | 106 ± 7 * |
| Left ventricular diastolic end | 4.1 ± 1.6 | 7.2 ± 4.0 * | 3.8 ± 2.1 | 4.6 ± 1.0 |
| pressure (mmHg) | | | | |
| Maximum left ventricular dp/dt | 13700 ± 1700 | 8300 ± 300 * | 9900 ± 1800 * † | 8300 ± 1000 * |
| (mmHg/s) | | | | |
| Minimum left ventricular dp/dt | −9700 ± 1600 | −6400 ± 700 * | −7500 ± 1300 * | −6400 ± 400 * |
| (mmHg/s) | | | | |

Data Notation: Average ± standard deviation
* P < 0.05 vs. Control SS (sham stimulation);
† P < 0.05 vs. I/R SS

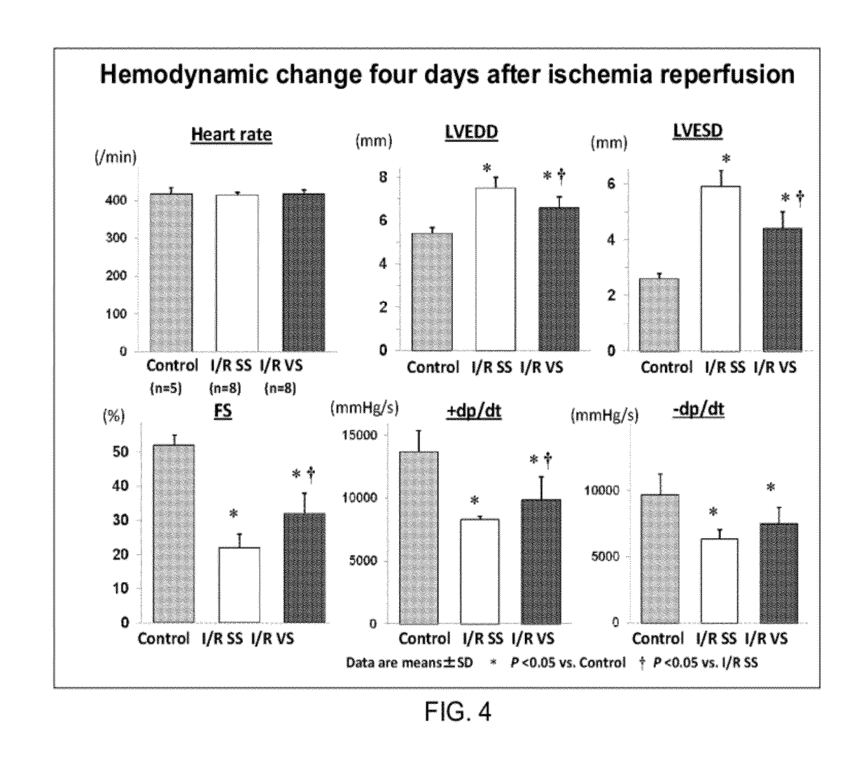

A graph of the results in Table 1 is illustrated in FIG. 4. A change in heart rate was seen, however, it is thought that stimulation of the vagus nerve significantly suppressed an increase in the left ventricle and a drop in the ejection rate that occur after ischaemia reperfusion, and suppressed myocardial remodeling.

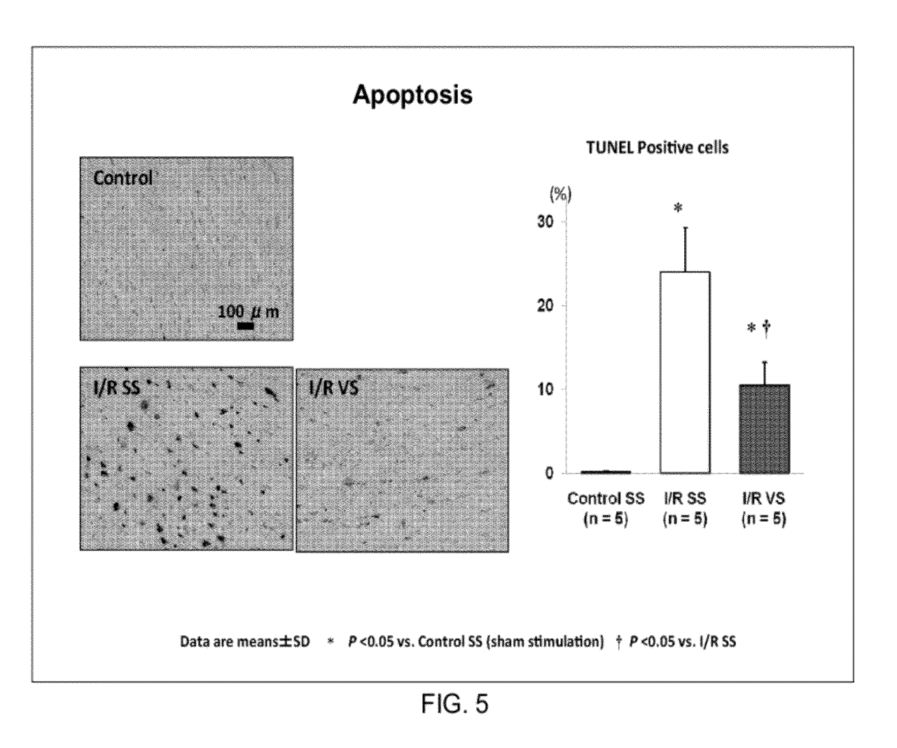

Furthermore, the appearance of apoptosis at the myocardial infarction site was studied as the mechanism of the anti-modeling effect due to vagus nerve stimulation (FIG. 5). Multiple TUNEL positive cells appear in the infarct area 24 hours after due to ischaemia reperfusion, however, it was found that these TUNEL positive cell were significantly suppressed in the myocardial infarction area after vagus nerve stimulation

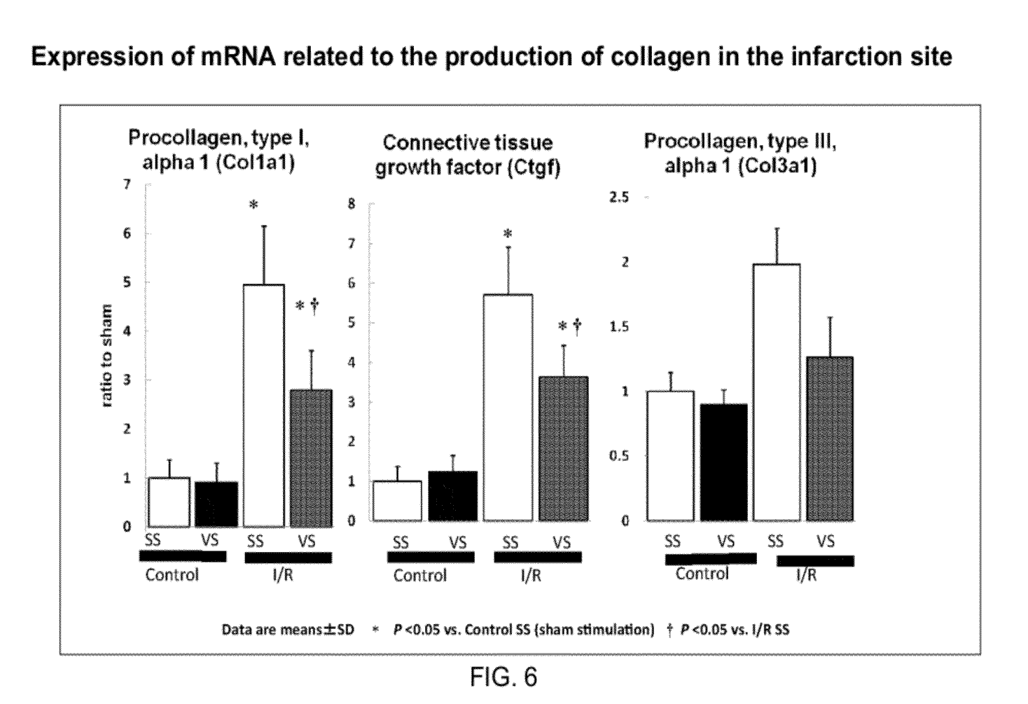

Moreover, when mRNA was collected from the myocardial infarction 3 hours after the myocardial infarction, and the amount of gene expression related to the generation of collagen was measured, it was evident that gene expression recognized as procollagen, type 1, type 3, and the connective tissue growth factor were significantly suppressed by vagus nerve stimulation. The results are illustrated in FIG. 6.

### CONCLUSION

It was shown that stimulation of the vagus nerve improves myocardial remodeling after ischaemia reperfusion and reduces the infarct area, and after that, hemodynamic improvement is obtained. The effect of suppressing apoptosis is considered to be one mechanism of that.

It is to be understood that the above-described embodiments are illustrative of only a few of the many possible specific embodiments that can represent applications of the principles of the invention. Numerous and varied other arrangements can be readily devised by those skilled in the art without departing from the spirit and scope of the invention.

1. An electrical stimulation device for treating cardiovascular disease comprising:
at least one electrode that is placed on a nerve site inside an animal; and
an electrical stimulation application unit to apply electrical stimulation to a vagus nerve in a neck region of said animal by said electrode.

2. The electrical stimulation device for treating cardiovascular disease according to claim 1, wherein
said cardiovascular disease is acute myocardial infarction.

3. The electrical stimulation device for treating cardiovascular disease according to claim 1, wherein
said electrical stimulation application unit applies electrical stimulation under conditions of a voltage of 0.1 to 5 V, a frequency of 1 to 30 Hz, and a pulse width of 500 μsec or greater.

4. The electrical stimulation device for treating cardiovascular disease according to claim 3, wherein
an amount of time that said electrical stimulation application unit applies electrical stimulation is at least 0.5 hour and no greater than 10 hours.

5. A method for treating cardiovascular disease in an animal comprising steps of:
placing an electrode in contact with a vagus nerve in a neck region of an animal; and
applying an electrical stimulation by said electrode.

6. The method for treating cardiovascular disease according to claim 5, wherein
said animal is a human.

7. The method for treating cardiovascular disease according to claim 5, wherein
said animal is an animal other than a human.

8. The method for treating cardiovascular disease according to claim 5, wherein
said cardiovascular disease is acute myocardial infarction.

9. The method for treating cardiovascular disease according to claim 8, wherein
said step of applying an electrical stimulation is used in combination with reperfusion therapy.

10. The method for treating cardiovascular disease according to claim 8, wherein
said step of applying an electrical stimulation is performed after an occurrence of acute myocardial infarction and before reperfusion therapy.

11. The method for treating cardiovascular disease according to claim 5, wherein
conditions for applying electrical stimulation are a voltage of 0.1 to 5 V, a frequency of 1 to 30 Hz, and a pulse width of 500 μsec or greater.

12. The electrical stimulation device for treating cardiovascular disease according to claim 2, wherein
said electrical stimulation application unit applies electrical stimulation under conditions of a voltage of 0.1 to 5 V, a frequency of 1 to 30 Hz, and a pulse width of 500 μsec or greater.

13. The electrical stimulation device for treating cardiovascular disease according to claim 12, wherein
an amount of time that said electrical stimulation application unit applies electrical stimulation is at least 0.5 hour and no greater than 10 hours.

14. The method for treating cardiovascular disease according to claim 6, wherein
conditions for applying electrical stimulation are a voltage of 0.1 to 5 V, a frequency of 1 to 30 Hz, and a pulse width of 500 μsec or greater.

15. The method for treating cardiovascular disease according to claim 7, wherein
conditions for applying electrical stimulation are a voltage of 0.1 to 5 V, a frequency of 1 to 30 Hz, and a pulse width of 500 μsec or greater.

16. The method for treating cardiovascular disease according to claim 8, wherein
conditions for applying electrical stimulation are a voltage of 0.1 to 5 V, a frequency of 1 to 30 Hz, and a pulse width of 500 μsec or greater.

17. The method for treating cardiovascular disease according to claim 9, wherein
conditions for applying electrical stimulation are a voltage of 0.1 to 5 V, a frequency of 1 to 30 Hz, and a pulse width of 500 μsec or greater.

18. The method for treating cardiovascular disease according to claim 10, wherein conditions for applying electrical stimulation are a voltage of 0.1 to 5 V, a frequency of 1 to 30 Hz, and a pulse width of 500 μsec or greater.
